(19) United States
(12) Patent Application Publication
(10) Pub. No.: US 2011/0230657 A1
(43) Pub. Date: Sep. 22, 2011

(54) PROCESS FOR PREPARING QUETIAPINE FUMARATE-TOLUENE-WATER

(75) Inventors: Ravi Ponnaiah, Vadodara (IN); Ashok Prasad, Vadodara (IN); Dineshkumar Ramabhai Panchasara, Vadodara (IN); Mitul Bipinchandra Patwa, Vadodara (IN); Ketan Bharatendubhai Patel, Vadodara (IN)

(73) Assignee: ALEMBIC LIMITED, GUJARAT, VADODARA (IN)

(21) Appl. No.: 13/130,961

(22) PCT Filed: Nov. 30, 2009
(86) PCT No.: PCT/IB09/55410

§ 371 Date: May 24, 2011

(30) Foreign Application Priority Data
Dec. 15, 2008 (IN) 2600/MUM/2008

(51) Int. Cl. C07D 417/04

(52) U.S. Cl. 540/551

(57) ABSTRACT

The present invention relates to an improved process for preparing Quetiapine fumarate of formula (I).

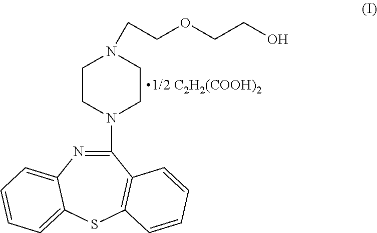

### FIELD OF THE INVENTION

The present invention relates to an improved process for preparing Quetiapine fumarate of formula (I).

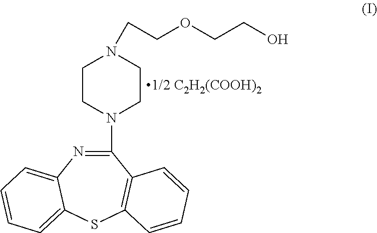

### BACKGROUND OF THE INVENTION

The chemical name of Quetiapine is 2-[2-(4-Dibenzo[b,f][1,4]thiazepin-11-yl-1-piperazinyl)ethoxy]ethanol and formula is C₂₁H₂₅N₃O₂S and molecular weight is 383.51. The drug is used in its fumarate salt. The current pharmaceutical product containing this drug is being sold by Astrazeneca using the tradename Seroquel and Seroquel XR, in the form of oral tablets.

Quetiapine is used as Antipsychotic. It is antipsychotic, psycholeptics and antidepressant class drug. It is dopamine antagonist and 5HT antagonist. It is used in the treatment of schizophrenia, bipolar diseases and psychosis. It is also used in the treatment of anxiety and depression.

U.S. Pat. No. 4,879,288 describes a process for the preparation of Quetiapine fumarate which is shown in the scheme-I.

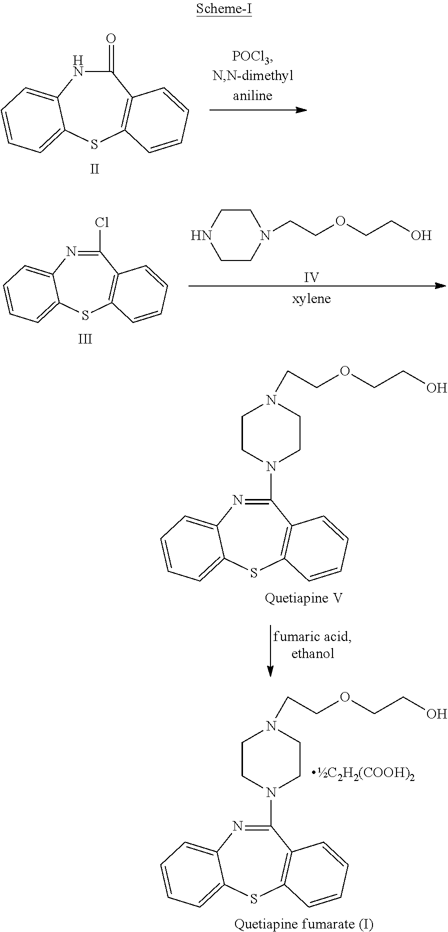

The process involves reacting dibenzo[b,f][1,4]thiazepine-11(10-H)-one (II) with phosphorous oxychloride in the presence of N,N-dimethylaniline to give imino chloride (III); which is further reacted with 1-(2-hydroxyethoxy)ethylpiperazine (IV) in xylene to give Quetiapine (V). Quetiapine is purified by flash chromatography and then converted into its fumarate salt by reacting it with fumaric acid in ethanol to give Quetaipine fumarate (I). The yield and purity obtained by this process is low. Further, this process requires chromatographic purification of Quetiapine before converting it to its fumarate salt. No purification process of Quetiapine fumarate is disclosed in this patent to enhance the purity of Quetiapine fumarate. In this process, the product obtained is having coloured impurity. Further it contains unknown impurity which is difficult to remove even after repeated purification.

It is therefore, a need to develop an easy to operate, industrially feasible process which also provides good yield and high purity of Quetiapine fumarate. The present invention addresses these needs.

Present inventors have directed their research work towards developing a process for the preparation of Quetiapine fumarate which provides good yield and purity. The present inventors observed that using toluene which contains little amount of water as solvent in condensation step of imino chloride (III) with 1-(2-hydroxyethoxy)ethylpiperazine (IV) instead of xylene increases yield and purity of quetiapine. It also improves the colour of the product. Further, the impurity which is generated in product patent does not generate by use of little amount of water along with toluene as solvent in this step. The observed that water amount is critical for this reaction step. The excess quantity of water with toluene makes the reaction slow. Therefore they optimized the quantity of water and found critical quantity of water which does not make the reaction sluggish; on the other hand it also prohibits the formation of impurity. The process eliminate the purification step by chromatography as mentioned in U.S. Pat. No. 4,879,288.

### SUMMARY OF THE INVENTION

Accordingly, it is an object of the present invention to provide a process for the preparation of Quetiapine fumarate.

Another object of the present invention is to provide a process which gives Quetiapine fumarate with high purity.

Another object of the present invention is to provide a process for the preparation of Quetiapine fumarate which is operationally simple and cost effective.

Accordingly, present invention provides a process for preparation of Quetiapine fumarate (I)

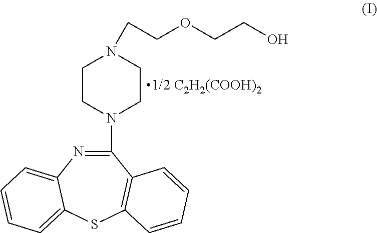

comprising a step of reacting 11-chlorodibenzo [b,f][1,4]thiazepine (III)

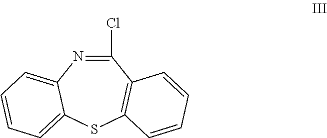

with 1-(2-hydroxyethoxy)ethylpiperazine (IV)

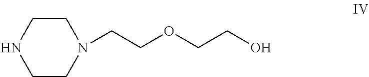

in the presence of a solvent system comprising toluene and water to give Quetiapine (V);

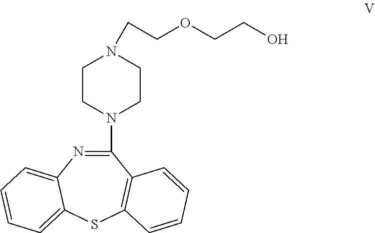

Accordingly, present invention provides a process for preparation of Quetiapine fumarate (I)

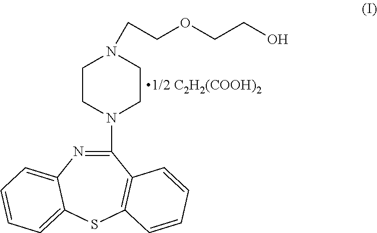

comprising steps of:
(i) dibenzo [b,f] [1,4] thiazepine-11(10-H)-one (II)

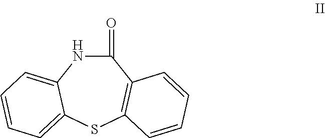

with phosphorous oxychloride in the presence of N,N-dimethylaniline in toluene to give 11-chlorodibenzo [b,f][1,4]thiazepine (III);

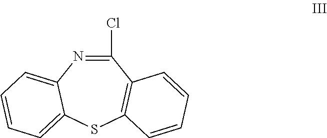

(ii) reacting 11-chlorodibenzo [b,f][1,4]thiazepine (III) with 1-(2-hydroxyethoxy)ethylpiperazine (IV)

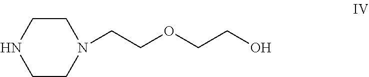

in the presence of a solvent system comprising toluene and water to give Quetiapine (V);

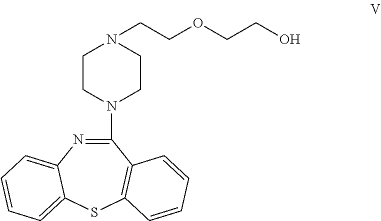

(iii) converting Quetiapine (V) to Quetiapine fumarate (I) by reacting it with fumaric acid in methanol.

The present invention provides a process for purification of Quetiapine fumarate (I) comprising steps of

(a) heating impure Quetiapine fumarate (I) with methanol
(b) adding water dropwise till clear solution obtained
(c) cooling the reaction mixture to obtain pure Quetiapine fumarate (I).

### DETAILED DESCRIPTION OF THE INVENTION

The present invention provides an improved process for preparation of Quetiapine fumarate (I)

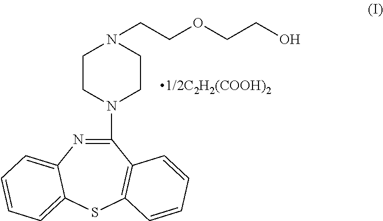

comprising steps of:
(i) dibenzo[b,f][1,4]thiazepine-11(10-H)-one (II)

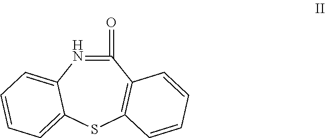

with phosphorous oxychloride in the presence of N,N-dimethylaniline in toluene to give 11-chlorodibenzo [b,f][1,4]thiazepine (III);

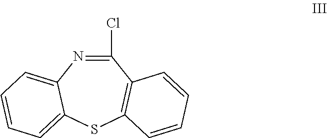

(ii) reacting 11-chlorodibenzo [b,f][1,4]thiazepine (III) with 1-(2-hydroxyethoxy)ethylpiperazine (IV)

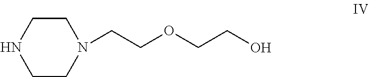

in the presence of a solvent system comprising toluene and water to give Quetiapine (V);

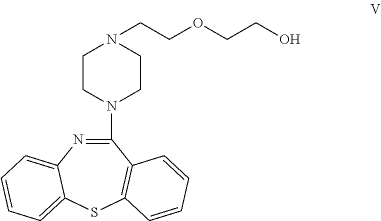

(iii) converting Quetiapine (V) to Quetiapine fumarate (I) by reacting it with fumaric acid in methanol.

The synthetic reaction scheme of the present invention is shown in the scheme-II.

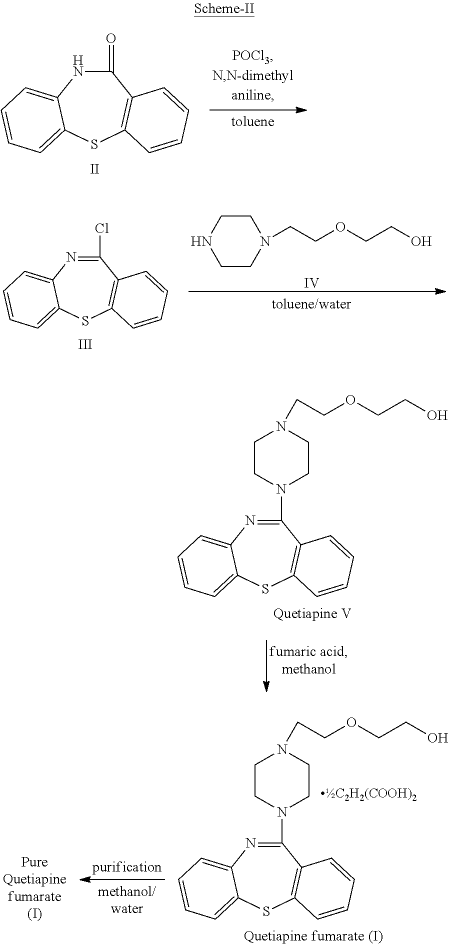

In the process of present invention, dibenzo[b,f][1,4]thiazepine-11(10-H)-one (II) is heated with Phosphorous oxychloride in the presence of N,N-Dimethyl aniline in toluene at about 110° C. to about 115° C. for 30 min. Toluene is distilled out from reaction mixture till half the original volume of reaction mixture remains. Cool the reaction mixture to 90° C. and add fresh toluene. Distill out toluene from the reaction mixture. This process is repeated. Again fresh toluene is added. The reaction mixture is cooled to 5° C. and chilled DM Water is added to the reaction mixture. Organic layer is separated and washed with water, aq. NaHCO₃ solution and aq. NaCl solution. Activated carbon is added to the organic layer and heated to 35° C. for 15 min. The reaction mixture is filtered through hyflo bed. The bed is washed with toluene. The filtrate and washings are combined and distilled out to remove solvent completely. The residue 11-chlorodibenzo [b,f][1,4]thiazepine (III) is obtained which is used as such for next step.

11-chlorodibenzo [b,f][1,4]thiazepine (III) is heated with 1-(2-hydroxyethoxy)ethylpiperazine (IV) in the presence of a solvent system comprising toluene and water at reflux temperature for about 8 hours. The ratio of water to toluene taken is in the range of about 1:30 to about 1:50. The progress of the reaction is monitored by HPLC. After completion of the reaction, the reaction mixture is cooled to 50-55° C. and quenched with DM Water. The organic layer is separated and aqueous layer is extracted with toluene. The combined organic layer is washed with DM Water and aq. NaCl solution. The organic layer is distilled out completely. Methanol is added to the residue and distilled out completely to give Quetiapine as viscous oil.

TABLE I

| | | INPUT (Chloro | | Hydroxy- | Colour of | |
|---|---|---|---|---|---|---|
| | Exp. | compound on basis | | Impurity | reaction | Product |
| Experiment | No. | of DBT input) | Solvent | generated (%) | mixture | Description |
| As per the | 1 | 100 g | Xylene | 0.23 | Dark Brown | Cream color |
| process | | | | | color | crystalline |
| known in | | | | | | powder |
| the literature | 2 | 50 g | Toluene | 0.17 | Dark Brown | Cream color |
| | | | | | color | crystalline |
| | | | | | | powder |
| Present | 3 | 10 g | Water:Toluene | 0.07 | Light yellow | White crystalline |
| invention | | | (1:70) | | color | powder |
| | 4 | 25 g | Water:Toluene | 0.06 | Light yellow | White crystalline |
| | | | (1:35) | | color | powder |
| | 5 | 25 g | Water:Toluene | 0.07 | Light yellow | White crystalline |
| | | | (1:14) | | color | powder |
| | 6 | 25 g | Water:Toluene | 0.08 | Light yellow | White crystalline |
| | | | (1:7) | | color | powder |

DBT is dibenzo[b,f][1,4]thiazepin-11(10H)-one

The present inventors have done the experiments varying the water:toluene ratio in condensation step. They also conducted the experiment without water according to the process known in the literature i.e. U.S. Pat. No. 4,879,288, Example-1. They observed that the hydroxyl impurity is which is generated in absence of water is reduced to great extent in the presence of water. The result is as shown in Table-I. Further, they also observed that when water:toluene ratio is taken in range of 1:30 to about 1:50, the impurity generated is minimum. The less amount of water will not effectively control the generation of impurity whereas more amount of water will sluggish the reaction. Therefore, the critical amount of water:toluene ratio is 1:30 to about 1:50, preferably 1:35. It is also observed that colour of the reaction also does not turn dark in the presence of water and which in turn also improves the colour of the product which is clearly seen in the above table.

Quetiapine is dissolved in methanol and heated to 45° C. till clear solution is obtained. Activated carbon is added and stirred for 15 min at 40-45° C. The mixture is cooled to 25° C. and filtered through hyflo bed. The bed is washed with methanol. The filtrate is heated to 60° C. A pre-dissolved solution of fumaric acid in methanol at about 60° C. to about 65° C. is added to preheated filtrate containing Quetiapine (V). The reaction mixture is further heated for 30 min at the same temperature. The reaction mixture is stirred for 4 hours at 20-25° C. The reaction mixture is cooled to about 5-10° C. and stirred for 1 hour. The separated solid is filtered, washed with chilled methanol and suck dried. The wet cake is used for purification.

The present invention provides a process for purification of Quetiapine fumarate (I) comprising steps of

(a) heating impure Quetiapine fumarate (I) with methanol;
(b) adding water dropwise till clear solution obtained;
(c) optionally reducing the volume of solution and;
(d) cooling the reaction mixture to obtain pure Quetiapine fumarate (I).

“Impure Quetiapine fumarate” means Quetiapine fumarate having HPLC purity less than 99.5%.

In the purification of Quetiapine fumarate, methanol is added to the wet cake of impure Quetiapine fumarate obtained in above process and heated at about 60° C. to about 65° C. DM Water is added drop wise at the same temperature till clear solution is obtained. The reaction mixture is stirred for 15-30 min at the same temperature. The mixture is filtered hot through hyflow bed. The bed is washed with hot methanol. Methanol from filtrate is recovered till half the original volume taken. The mixture is cooled to about 20-25° C. and stirred for 4 hours. It is further cooled to 0° C. to about 5° C. and stirred for 1 hour. The separated solid is filtered, washed with chilled methanol and suck dried. The solid is dried under vacuum at about 50° C. to about 55° C. to give pure Quetiapine fumarate (I)

The following examples illustrate the invention further. It should be understood, however, that the invention is not confined to the specific limitations set forth in the individual examples but rather to the scope of the appended claims.

### Example-1

### Preparation of 11-chlorodibenzo [b,f][1,4]thiazepine (III)

dibenzo[b,f][1,4]thiazepine-11(10-H)-one (II) (100 g) was heated with phosphorous oxychloride (202 g) in the presence of N,N-Dimethyl aniline (79.97 g) in toluene (800 ml) at 110° C. to 115° C. for 30 min. Toluene is distilled out from reaction mixture till 400-450 ml residual volume of reaction mixture remains. Cool the reaction mixture to 90° C. and add fresh toluene (400 ml). Distill out toluene from the reaction mixture at 110° C. till 400-450 ml residual volume of reaction mixture remains. This process is repeated. Again fresh toluene (400 ml) is added. Monitor the reaction on HPLC. The reaction mixture is cooled to 5° C. and chilled DM Water (400 ml) is added to the reaction mixture and stirred for 30 min at 20-25° C. Organic layer is separated and washed with DM Water (200 ml), aq. NaHCO₃ solution (8 g NaHCO₃ in 96 ml DM Water) and aq. NaCl solution (40 g in 180 ml DM Water). Activated carbon (5 g) is added to the organic layer and heated to 35° C. for 15 min. The reaction mixture is filtered through hyflo bed. The bed is washed with toluene (100 ml). The filtrate and washings are combined and distilled out to remove solvent completely. The residue 11-chlorodibenzo [b,f][1,4]thiazepine (III) obtained is used as such for next step.

### Example-2

### Preparation of Quetiapine Base (V)

Residue 11-chlorodibenzo [b,f][1,4]thiazepine (III) obtained from Example-1 was heated with 1-(2-hydroxyethoxy)ethylpiperazine (IV) (160.99 g) in the presence of toluene (700 ml) and water (20 ml) at reflux temperature for about 8 hours. The progress of the reaction was monitored by HPLC. After completion of the reaction, the reaction mixture is cooled to 50-55° C. and quenched with DM Water (200 ml). The organic layer is separated and aqueous layer is extracted with toluene (100 ml). The combined organic layer is washed with DM Water (200 ml×2) and aq. NaCl solution (40 g in 180 ml DM Water). The organic layer is distilled out completely. Methanol is added to the residue and distilled out completely to give Quetiapine as viscous oil which is used for next step.

### Example-3

### Preparation of Quetiapine Fumarate (I)

Quetiapine obtained in Example-2 is dissolved in methanol (250 ml) and heated to 45° C. till clear solution was obtained. Activated carbon (5 g) is added and stirred for 15 min at 40-45° C. The mixture is cooled to 25° C. and filtered through hyflo bed. The bed is washed with methanol (100 ml). The filtrate is heated to 60° C. A pre-dissolved solution of fumaric acid (26.55 g) in methanol (320 ml) at about 60° C. to about 65° C. is added to preheated filtrate containing Quetiapine (V). The reaction mixture is further heated for 30 min at the same temperature. The reaction mixture is stirred for 4 hours at 20-25° C. The reaction mixture is cooled to about 5-10° C. and stirred for 1 hour. The separated solid is filtered, washed with chilled methanol (50 ml×2) and suck dried. And dried in oven under vacuum at 60-65° C. to give Quetiapine Furmarate (120-140 g)

Yield: 1.2-1.4 (w/w)

Purity (by HPLC): >98%

### Example-4

### Purification of Quetiapine Fumarate (I)

Quetiapine fumarate (100 g) obtained from Example-3 was heated in methanol (1000 ml) at 65° C. to 70° C. DM Water (30 ml) is added drop wise at the same temperature till clear solution is obtained. The reaction mixture is stirred for 15-30 min at the same temperature. The mixture is filtered hot through hyflow bed. The bed is washed with hot methanol (100 ml). Methanol from filtrate is recovered till residual volume is 550-600 ml remains. The mixture is cooled to about 20-25° C. and stirred for 4 hours. It is further cooled to about 0-5° C. and stirred for 1 hour. The separated solid is filtered, washed with chilled methanol (100 ml) and suck dried. The solid is dried under vacuum at about 50° C. to about 55° C. to give pure Quetiapine fumarate (I) (85-90 g)

Yield: 0.85-0.90 (w/w)

Purity (by HPLC): 99.5%

What is claimed is:

1. A process for preparation of Quetiapine fumarate (I) 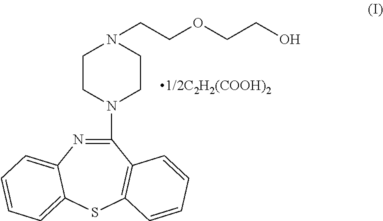
comprising a step of reacting 11-chlorodibenzo [b,f][1,4]thiazepine (III) 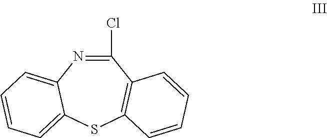
with 1-(2-hydroxyethoxy)ethylpiperazine (IV) 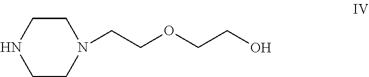
in the presence of a solvent system comprising toluene and water to give Quetiapine (V);

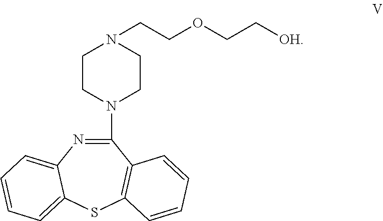

2. The process as claimed in claim 1, wherein ratio of water:toluene taken is in the range of about 1:30 to about 1:50.

3. The process as claimed in claim 1, wherein ratio of water:toluene taken is about 1:35.

4. A process for preparation of Quetiapine fumarate (I) 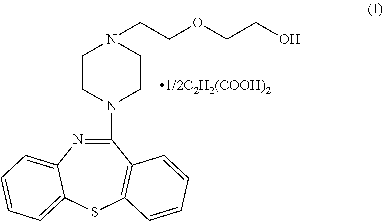
comprising steps of:
(i) dibenzo [b,f] [1,4]thiazepine-11(10-H)-one (II) 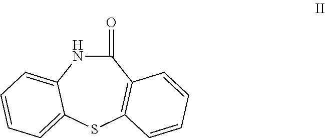
with phosphorous oxychloride in the presence of N,N-dimethylaniline in toluene to give 11-chlorodibenzo [b,f][1,4]thiazepine (III); 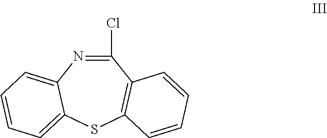
(ii) reacting 11-chlorodibenzo [b,f][1,4]thiazepine (III) with 1-(2-hydroxyethoxy)ethylpiperazine (IV) 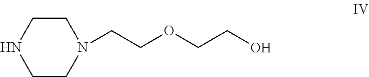
in the presence of a solvent system comprising toluene and water to give Quetiapine (V); 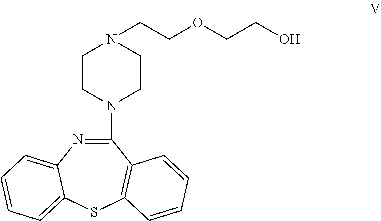
(iii) converting Quetiapine (V) to Quetiapine fumarate (I) by reacting it with fumaric acid in methanol.

5. The process as claimed in claim 4, wherein ratio of water:toluene taken is in step (ii) is in the range of about 1:30 to about 1:50.

6. A process for purification of Quetiapine fumarate (I) comprising steps of
(a) heating impure Quetiapine fumarate (I) with methanol;
(b) adding water dropwise till clear solution obtained;
(c) optionally reducing the volume of solution and;
(d) cooling the reaction mixture to obtain pure Quetiapine fumarate (I).
